(19) United States
(12) Patent Application Publication
(10) Pub. No.: US 2003/0009122 A1
(43) Pub. Date: Jan. 9, 2003

(54) Visi-band

(76) Inventor: Harry George Veras, River Edge, NJ (US)

Correspondence Address: Harry G. Veras, 124 Reservoir Avenue, Apt. 1B, River Edge, NJ, 07661, US

(21) Appl. No.: 09/985,234

(22) Filed: Nov. 2, 2001

Related U.S. Application Data
(60) Provisional application No. 60/243,838, filed on Oct. 30, 2000.

(51) Int. Cl. A61F 13/00

(52) U.S. Cl. 602/42; 602/54

(57) ABSTRACT

Visi-Band is a new concept for treating cuts, scrapes, wounds, small surgical incisions and other relatively small damaged areas on the human body. The concept of use and the design of the bandage are completely opposite from present day practice. Whereas, today, bandages are removed on a regular basis, perhaps daily or more frequently, with Visi-Band, the bandage stays in place until the wound, etc. has healed. This is accomplished by using a plastic cover with an opening lid applied to the area with self-adhesive and merely opening the cover as needed to treat the wound inside. Just a few of the many advantages, include protection of the wound from any further damage from such problems as banging into it; the ability to provide much more healing air to the area; the ability to apply pressure to the wound; the ease with which daily medication may be accomplished. The surrounding area is not subject to the problems of removing adhesive tape every time the wound is dressed.

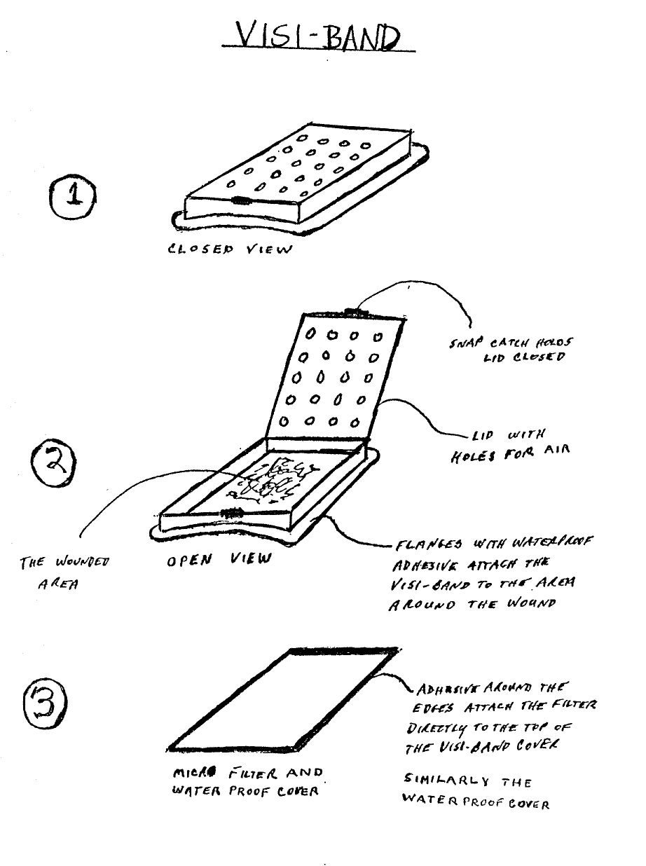

### CROSS REFERENCE TO RELATED APPLICATIONS

Not Applicable.

### STATEMENT REGARDING FEDERALLY SPONSORED RESEARCH OR DEVELOPMENT

Not Applicable.

### REFERENCE TO MICROFICHE APPENDIX

Not Applicable.

### BACKGROUND OF THE INVENTION

This invention pertains to the field of medicine and specifically to a new method of treating wounds, scrapes, cuts, small surgical incisions and boils, blisters and other similar maladies. This new concept and methodology was invented to improve upon the way in which such wounds are treated and to alleviate the problems encountered by the present methodology. Until now, the generally accepted methodology for treating such cuts, wounds, etc. has been to apply various materials directly to the affected area. Some of the problems encountered by using the current technology are the sticking of the bandage to the wound causing damage upon removal, damage to the surrounding skin by daily or more frequent removal of adhesive tape, reduced amounts of air reaching the wound thus slowing healing, and of significant difference, the failure of the current methods to protect the wound from accidentally bumping it, knocking into something or having clothing or other things rub it.

### BRIEF SUMMARY OF THE INVENTION

The invention is made of clear crush resistant plastic and should be visualized as a small plastic box with a snap-open/close top and no bottom. Instead of a bottom it has small flanges turning ninety degrees outward from the four sides, containing waterproof adhesive to attach it around the affected area. It would be available in many sizes and shapes so as to meet any particular need. It is affixed to the affected area with waterproof adhesive and remains in place until the wound is healed. Its snap-open/close top allows for application of cleaning solutions, antibiotics or any other medication needed, all without removing the unit. When finished with the periodic treating of the wound, the cover snaps back closed. The invention thus avoids the problems enumerated above by providing a protective shield to the area involved (no bumping, scraping or clothing rubbing on it), by not placing any material directly on the wound, unless advised to do so by the physician, (thus avoiding damage caused by sticking dressings), by avoiding any damage to the surrounding area, often times tender and painful in and of itself, by the constant removal of the bandage (Visi-Band remains on until the wound has healed.) and in addition allowing large amounts of air to circulate around the wound. Another plus is the fact that the monitoring of the progress of the wound may be done as frequently as desired, just by looking at it without removing a bandage.

### BRIEF DESCRIPTION OF SEVERAL VIEWS OF THE DRAWING

View number 1 on the drawing shows the Visi-Band in the closed position as it would appear, for example, on the patient's leg, arm or other body area

View number 2 on the drawing shows the same unit with the lid open allowing for treatment of the wounded area.

View number 3 on the drawing shows a depiction of both a micro filter and a waterproof cover, each of which can be attached or removed from the visi-band as needed.

### DETAILED DESCRIPTION OF THE INVENTION

Visi-Band was invented to provide a substantial number of distinct advantages, which are not presently in use, in the treatment of wounds, cuts, bruises and small surgical incisions. It is, in effect, a new way of bandaging wounds as we now do, without using bandages as we know them. It is a plastic shield affixed around the area, which stays in place until the wound is completely healed or requires no further protection. Some of the advantages are:

The treated area is protected from further damage by being banged into, scraped or in any other way touched unintentionally.

The area around the wound, which in many instances is tender and made uncomfortable by the frequent changing of adhesive bandages, has the Visi-Band attached only once with waterproof adhesive, and does not come off until the wound has healed.

In many instances, the wound is covered with a bandage just to protect it from additional damage, but in the process, much of the air is deleted from it. The air is desirable to aid in the healing process. With the Visi-Band, unless otherwise indicated, the wound is left open because it is protected by the Visi-Band. The result is, with the holes in the lid of the Visi-Band, the wound, in effect, is left completely open to the air and yet completely protected. This also allows for the complete inspection of the wound at any time, merely by looking in or opening the cover.

In those situations where it is desirable to have as much air as possible, but it is necessary to keep any foreign matter in the air away from the wound, the very thin self adhesive micro filter pad is affixed to the cover of the Visi-Band, thus filtering the air that enters. In addition, a self adhesive micro filter pad, treated with anti-biotic powder, may be used, thus further purifying the air entering the wounded area.

In some instances, at the outset, for various reasons, it may be necessary to apply pressure to the wound. This can be easily accomplished by first cleaning and medicating the wound, secondly applying the Visi-Band and finally by applying non stick pads to the wound, with gauze pads or even cotton balls on top, until the Visi-Band is full and then closing the lid. Thus, any desired amount of pressure can be applied by putting more or less gauze or cotton balls inside. When the need for the pressure ceases, all that need be done is to remove the gauze, etc. form inside the Visi-Band. (A Visi-Band without holes in the lid will also be available for the application of wet compresses.)

One very important plus to the Visi-Band is the ability to place a waterproof self-adherent cover over the holes in the lid thus making the unit completely waterproof. In the past, patients have been prevented from bathing or showering for any number of days because of the necessity of keeping the bandage dry. No longer a problem with Visi-Band.

What I claim as my invention is:

1. A totally new concept and device for treating wounds, cuts, scrapes, boils, blisters, small surgical incisions and other relatively small damages to the outer human body. This concept and the device used to carry it out are exactly and completely opposite to what has been done now for years and years. Currently bandages are applied directly to wounds, etc. The Visi-Band concept and application do just the opposite. I have invented a device to carry out this totally different methodology and provide all the additional benefits that accrue from using it. What I consider to be patentable is the device itself and the name I have given it.
